(19) United States
(12) Patent Application Publication
(10) Pub. No.: US 2025/0204926 A1
(43) Pub. Date: Jun. 26, 2025

(54) CONTROLLED SHIELDING OF SHAPE MEMORY POLYMERS AND FOAMS

(71) Applicant: BOSTON SCIENTIFIC SCIMED, INC., MAPLE GROVE, MN (US)

(72) Inventors: Patrick Willoughby, Shoreview, MN (US); Tatyana Dyndikova, Fort Worth, TX (US)

(73) Assignee: BOSTON SCIENTIFIC SCIMED, INC., MAPLE GROVE, MN (US)

(21) Appl. No.: 18/987,989

(22) Filed: Dec. 19, 2024

Related U.S. Application Data
(60) Provisional application No. 63/612,493, filed on Dec. 20, 2023.
(60) Provisional application No. 63/612,507, filed on Dec. 20, 2023.
(60) Provisional application No. 63/612,569, filed on Dec. 20, 2023.
(60) Provisional application No. 63/612,582, filed on Dec. 20, 2023.
(60) Provisional application No. 63/561,406, filed on Mar. 5, 2024.
(60) Provisional application No. 63/561,415, filed on Mar. 5, 2024.
(60) Provisional application No. 63/560,160, filed on Mar. 1, 2024.
(60) Provisional application No. 63/560,174, filed on Mar. 1, 2024.

(51) Int. Cl. A61B 17/12; A61B 17/00

(52) U.S. Cl. CPC A61B 17/12172; A61B 2017/00004; A61B 2017/00836; A61B 2017/00871; A61B 2017/00898; A61B 2017/00938; A61B 2017/00942

(57) ABSTRACT

An occlusive implant includes an expandable foam member in a compressed configuration and a biodegradable capsule disposed over the expandable foam member. The expandable foam member is made of a shape memory polymer, and the biodegradable capsule holds the expandable foam member in the compressed configuration for a first time period, after which the biodegradable capsule degrades, allowing the expandable form member to expand into an expanded configuration.

### CROSS-REFERENCE TO RELATED APPLICATIONS

This application claims the benefit of priority of U.S. Provisional Application No. 63/612,493 filed Dec. 20, 2023, 63/612,507, filed Dec. 20, 2023, 63/612,569, filed Dec. 20, 2023, 63/612,582, filed Dec. 20, 2023, 63/561,406, filed Mar. 5, 2024, 63/561,415, filed Mar. 5, 2024, 63/560,160, filed Mar. 1, 2024, and 63/560,174, filed Mar. 1, 2024, the entirety disclosure of which is hereby incorporated by reference.

### TECHNICAL FIELD

The disclosure relates generally to medical devices and more particularly to medical devices that are adapted for use in percutaneous medical procedures in which an expandable shape memory material is inserted into the body.

### BACKGROUND

A wide variety of medical devices have been developed for medical use including, for example, medical devices utilized to occlude regions of the body. These medical devices may be used in a variety of body regions including an aneurysm in a vessel and the left atrial appendage (LAA). In patients suffering from atrial fibrillation, the LAA may not properly contract or empty, causing stagnant blood to pool within its interior, which can lead to the undesirable formation of thrombi within the LAA.

Thrombi forming in the LAA may break loose from this area and enter the blood stream. Thrombi that migrate through the blood vessels may eventually plug a smaller vessel downstream and thereby contribute to stroke or heart attack. Clinical studies have shown that the majority of blood clots in patients with atrial fibrillation originate in the LAA. As a treatment, medical devices have been developed which are deployed to close off the LAA. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example occlusive implant includes an expandable foam member in a compressed configuration, the expandable foam member made of a shape memory polymer, and a biodegradable capsule disposed over the expandable foam member, the biodegradable capsule holding the expandable foam member in the compressed configuration for a first time period, after which the biodegradable capsule degrades, allowing the expandable foam member to expand into an expanded configuration.

Alternatively or additionally to the embodiment above, the biodegradable capsule covers an entirety of an outer surface of the expandable foam member.

Alternatively or additionally to any of the embodiments above, the biodegradable capsule includes at least one aperture extending through a wall of the biodegradable capsule.

Alternatively or additionally to any of the embodiments above, when in the compressed configuration, the expandable foam member and the biodegradable capsule are elongate with first and second opposing ends, wherein the biodegradable capsule covers all but the second end of the expandable foam member.

Alternatively or additionally to any of the embodiments above, when in the compressed configuration, the expandable foam member and the biodegradable capsule are elongate with first and second opposing ends, wherein the biodegradable capsule includes a plurality of spaced apart apertures disposed adjacent the first end.

Alternatively or additionally to any of the embodiments above, the first time period is a preset time period.

Alternatively or additionally to any of the embodiments above, the preset time period is 30 seconds to 4 minutes.

Alternatively or additionally to any of the embodiments above, when in the compressed configuration, the expandable foam member is elongate with first and second opposing ends, the expandable foam member having a first dimension extending between the first and second opposing ends, and a second dimension transverse to the first dimension, the first dimension being longer than the second dimension, wherein when the expandable foam member is in the expanded configuration, the first dimension is shorter than the second dimension.

Alternatively or additionally to any of the embodiments above, when in the expanded configuration the first dimension is between 3-15 mm and the second dimension is between 20-35 mm.

Alternatively or additionally to any of the embodiments above, the biodegradable capsule is made of one or more materials selected from the group consisting of poly (ethylene glycol), polyvinyl alcohol, polyurethane, polylactic acid, poly(lactide-co-glycolide), poly(ε-caprolactone), sugar derivatives, salt, high surface area electrospun materials, and hydrogels.

Alternatively or additionally to any of the embodiments above, the biodegradable capsule is made of a copolymer of a hydrophobic component and a hydrophilic component.

Alternatively or additionally to any of the embodiments above, the copolymer includes poly(ethylene glycol) and polylactic acid.

Alternatively or additionally to any of the embodiments above, the biodegradable capsule incudes a first section and a second section sealed to the first section.

Alternatively or additionally to any of the embodiments above, the occlusive implant further includes an expandable framework configured to shift between a collapsed configuration and an expanded configuration, the expandable framework defining an interior, wherein the expandable foam member is disposed within the interior.

Alternatively or additionally to any of the embodiments above, when the expandable framework is in the expanded configuration and the biodegradable capsule has degraded, the expandable foam member expands to fill an entirety of at least a proximal region of the expandable framework.

Alternatively or additionally to any of the embodiments above, the occlusive implant further includes an occlusive covering disposed on a proximal end region of the expandable framework.

Another example occlusive implant includes an expandable foam member in a compressed configuration, the expandable foam member made of a shape memory polymer that expands when exposed to water, increased temperature, change in pH, or electrical stimulation, and a biodegradable capsule disposed over the expandable foam member, the biodegradable capsule holding the expandable foam member in the compressed configuration for a first time period, after which the biodegradable capsule degrades, allowing the expandable foam member to expand into an expanded configuration, wherein when in the compressed configuration, the expandable foam member is elongate with first and second opposing ends, the expandable foam member having a first dimension extending between the first and second opposing ends, and a second dimension transverse to the first dimension, the first dimension being longer than the second dimension, wherein when the expandable foam member is in the expanded configuration, the first dimension is shorter than the second dimension.

Alternatively or additionally to the embodiments above, the occlusive implant further includes an expandable framework configured to shift between a collapsed configuration and an expanded configuration, the expandable framework defining an interior, wherein the expandable foam member is disposed within the interior.

Alternatively or additionally to any of the embodiments above, when the expandable framework is in the expanded configuration and the biodegradable capsule has degraded, the expandable foam member expands to fill an entirety of at least a proximal region of the expandable framework.

An example method of occluding a body cavity includes the steps of inserting an expandable foam member in a compressed configuration into a self-expandable framework, the expandable foam member made of a shape memory polymer and covered by a biodegradable capsule, the biodegradable capsule holding the expandable foam member in the compressed configuration, the self-expandable framework configured to shift between a collapsed configuration and an expanded configuration, compressing the self-expandable framework to the collapsed configuration with the expandable foam member inside, delivering the self-expandable framework to a body cavity, expanding the self-expandable framework, and degrading the biodegradable capsule in the presence of moisture and/or heat within the body cavity, thereby expanding the expandable foam member, wherein the expandable foam member expands to fill at least a proximal region of the expanded self-expandable framework.

The above summary of some embodiments, aspects, and/or examples is not intended to describe each embodiment or every implementation of the present disclosure. The figures and the detailed description which follows more particularly exemplify these embodiments.

### BRIEF DESCRIPTION OF THE DRAWINGS

The disclosure may be more completely understood in consideration of the following detailed description of various embodiments in connection with the accompanying drawings, in which:

FIGS. 1-2 are side views illustrating selected aspects of an occlusive implant system for occluding a left atrial appendage;

FIGS. 3-4 illustrate selected aspects of an occlusive implant for occluding a left atrial appendage;

FIGS. 5A and 5B are cross-sectional views of an example occlusive implant in the compressed and expanded configurations, respectively;

FIGS. 6A and 6B are cross-sectional views of another example occlusive implant in the compressed and partially expanded configurations, respectively;

FIG. 7A is a side view of a further example of an occlusive implant;

FIGS. 7B and 7C are cross-sectional views of the occlusive implant of FIG. 7A taken along int 7B-7B, in the compressed and partially expanded configurations, respectively;

FIG. 8 is a cross-sectional view of another example occlusive implant;

FIG. 9A is a side cross-sectional view of another example occlusive implant in a delivery sheath; and

FIG. 9B is a side cross-sectional view of the occlusive implant of FIG. 9B in a deployed, expanded configuration.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “withdraw”, variants thereof, and the like, may be generally considered with respect to the positioning, direction, and/or operation of various elements relative to a user/operator/manipulator of the device, wherein “proximal” and “withdraw” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc. Additionally, the term “substantially” when used in reference to two dimensions being “substantially the same” shall generally refer to a difference of less than or equal to 5%.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to affect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

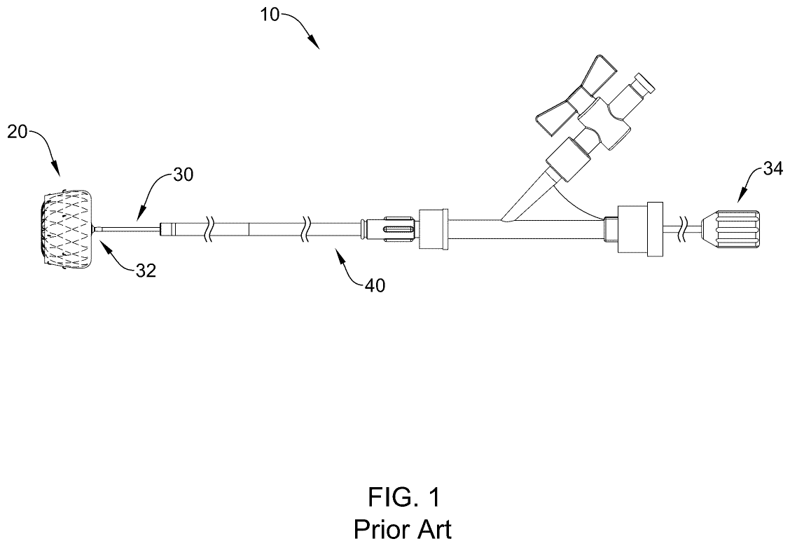

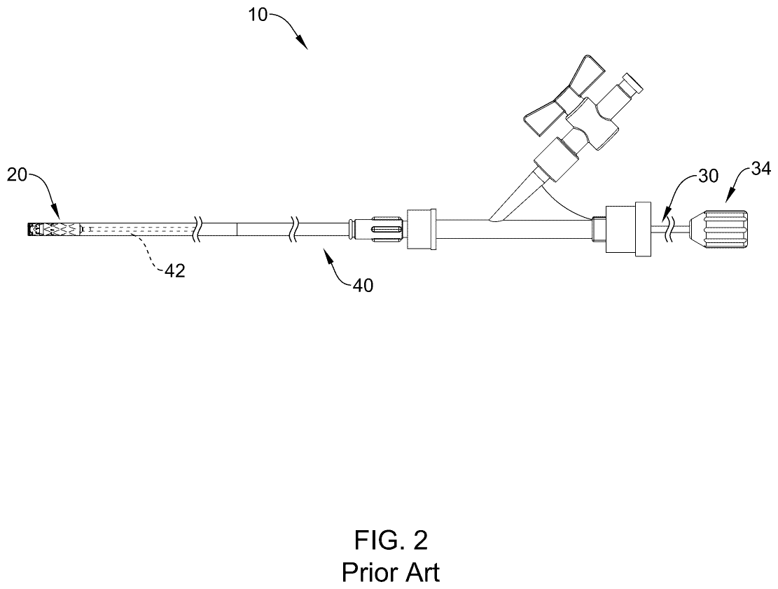

FIGS. 1-2 illustrate selected components and/or arrangements of a prior art occlusive implant system 10 for occluding a left atrial appendage. It should be noted that in any given figure, some features of the occlusive implant system 10 may not be shown, or may be shown schematically, for simplicity. Additional details regarding some of the components of the occlusive implant system 10 may be illustrated in other figures in greater detail. The occlusive implant system 10 may be used to percutaneously deliver and/or deploy a variety of medical implants (e.g., a cardiovascular implant, an occlusive implant, a replacement heart valve implant, etc.) to one or more locations within the anatomy, including but not limited to, in some embodiments, the heart.

The occlusive implant system 10 may comprise a core wire 30. The occlusive implant system 10 may comprise an occlusive implant 20 releasably coupled to and/or disposed at a distal end 32 of the core wire 30. In at least some embodiments, the occlusive implant 20 may be configured to occlude the left atrial appendage. The left atrial appendage is attached to and in fluid communication with the left atrium of the patient's heart. The left atrial appendage may have a complex geometry and/or irregular surface area.

In some embodiments, the occlusive implant system 10 may include a delivery sheath 40 having a lumen 42 (e.g., FIG. 2) extending from a proximal opening to a distal opening. In some embodiments, the core wire 30 may be slidably disposed within the lumen 42 of the delivery sheath 40. The core wire 30 may have a proximal end 34 disposed proximal of the delivery sheath 40. In some embodiments, the proximal end 34 of the core wire 30 may include a knob and/or a handle configured to manipulate and/or move the core wire 30 and/or the occlusive implant 20. In some embodiments, the proximal end 34 of the core wire 30 may include a knob and/or a handle configured to manipulate and/or move the core wire 30 and/or the occlusive implant 20 relative to the delivery sheath 40. In some embodiments, the delivery sheath 40 may be sized and configured to deliver the occlusive implant 20 to the left atrial appendage.

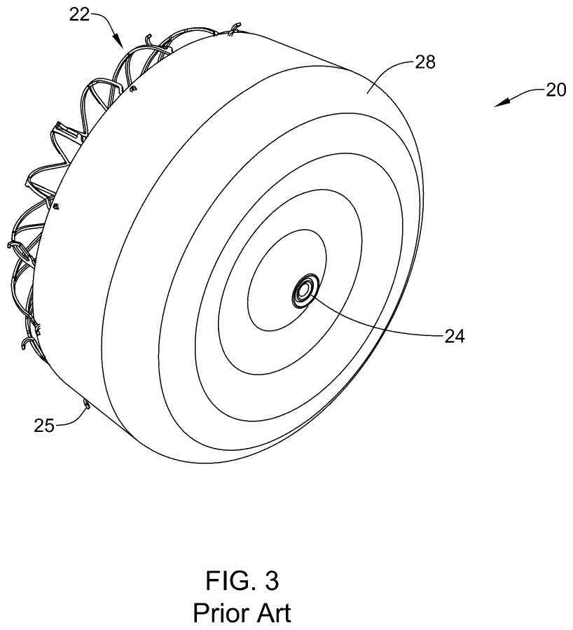

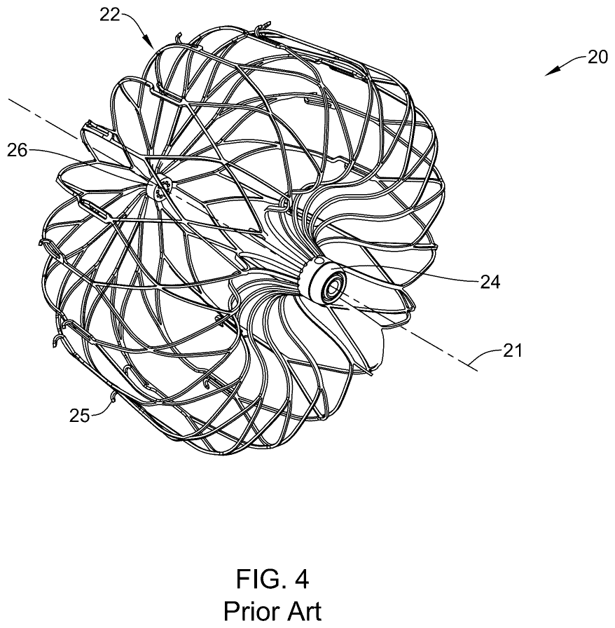

The occlusive implant 20 may include an expandable framework 22 (e.g., FIGS. 3-4) configured to shift between a collapsed configuration (e.g., FIG. 2) and a deployed configuration (e.g., FIG. 1). In some embodiments, the occlusive implant 20 may be disposed within the lumen 42 proximate the distal opening in the collapsed configuration. In some embodiments, the delivery sheath 40 may constrain the occlusive implant 20 and/or the expandable framework 22 in the collapsed configuration. In some embodiments, the occlusive implant 20 and/or the expandable framework 22 may be configured to shift between the collapsed configuration and the deployed configuration when the occlusive implant 20 is disposed distal of the distal opening of the lumen 42 and/or the delivery sheath 40, and/or when the occlusive implant 20 is unconstrained. In some embodiments, the occlusive implant 20 and/or the expandable framework 22 may be configured to shift between the collapsed configuration and the deployed configuration when the occlusive implant 20 is unconstrained by the delivery sheath 40. In at least some embodiments, the expandable framework 22 may be self-biased toward the deployed configuration.

In some embodiments, the core wire 30 may be slidably and/or rotatably disposed within the lumen 42 of the delivery sheath 40. In some embodiments, the proximal end 34 of the core wire 30 may extend proximally of a proximal end of the delivery sheath 40 and/or the proximal opening of the lumen 42 for manual manipulation by a clinician or practitioner. In some embodiments, the occlusive implant 20 may be removably attached, joined, secured, or otherwise connected to the distal end 32 of the core wire 30. The core wire 30 may be configured to and/or may be capable of axially translating the occlusive implant 20 relative to the delivery sheath 40. The delivery sheath 40 and/or the core wire 30 may have a selected level of axial stiffness and/or pushability characteristics while also having a selected level of flexibility to permit navigation through the patient's vasculature.

Some suitable, but non-limiting, examples of materials for the occlusive implant system 10, the core wire 30, the delivery sheath 40, and/or the occlusive implant 20, etc. are discussed below.

FIGS. 3 and 4 illustrate selected components and/or arrangements of a prior art occlusive implant 20 for occluding a left atrial appendage. The occlusive implant 20 may comprise an expandable framework 22 configured to shift along a longitudinal axis 21 (e.g., FIG. 4) between the collapsed configuration and the deployed configuration. In the collapsed configuration, the expandable framework 22 may be axially elongated and/or radially compressed. In the deployed configuration, the expandable framework 22 may be axially shortened and/or radially expanded. The expandable framework 22 may comprise a plurality of interconnected struts defining a plurality of cells. In some embodiments, the plurality of cells may be a plurality of closed cells. In some embodiments, the plurality of cells may be a plurality of open cells. In some embodiments, the plurality of cells may include a plurality of open cells and a plurality of closed cells in various combinations and/or arrangements. In some embodiments, the plurality of interconnected struts may converge, join, and/or connect at intersections or nodes.

The plurality of interconnected struts may be formed and/or cut from a tubular member. In some embodiments, the plurality of interconnected struts may be integrally formed and/or cut from a unitary member. In some embodiments, the plurality of interconnected struts may be integrally formed and/or cut from a unitary tubular member and subsequently formed and/or heat set to a desired shape in the deployed configuration. In some embodiments, the plurality of interconnected struts may be integrally formed and/or cut from a unitary flat member or sheet, and then rolled or formed into a tubular structure and subsequently formed and/or heat set to the desired shape in the deployed configuration. Some exemplary means and/or methods of making and/or forming the plurality of interconnected struts include laser cutting, machining, punching, stamping, electro discharge machining (EDM), chemical dissolution, etc. Other means and/or methods are also contemplated.

In some embodiments, the expandable framework 22 may be compliant and substantially conform to and/or be in sealing engagement with the shape and/or geometry of a wall of the left atrial appendage in the deployed configuration. In some embodiments, the occlusive implant 20 may expand to a size, extent, or shape less than or different from a maximum unconstrained extent, as determined by the surrounding tissue and/or wall of the left atrial appendage. In some embodiments, reducing a thickness of various elements of the expandable framework 22 may increase the flexibility and compliance of the expandable framework 22 and/or the occlusive implant 20, thereby permitting the expandable framework 22 and/or the occlusive implant 20 to conform to the tissue around it, rather than forcing the tissue to conform to the expandable framework 22 and/or the occlusive implant 20. In some embodiments, the expandable framework 22 and/or the occlusive implant 20 may be stronger and/or less compliant, and thus the expandable framework 22 and/or the occlusive implant 20 may force the tissue of the left atrial appendage to conform to the expandable framework 22 and/or the occlusive implant 20 in the deployed configuration. Other configurations are also contemplated.

In some embodiments, the occlusive implant 20 and/or the expandable framework 22 may comprise a plurality of anchoring elements 25. In some embodiments, the plurality of anchoring elements 25 may extend radially outward from the expandable framework 22 in the deployed configuration. In at least some embodiments, the plurality of anchoring elements 25 may be configured to engage with tissue and/or may be configured to secure the occlusive implant 20 and/or the expandable framework 22 to tissue at a target site (e.g., the left atrial appendage, etc.). In some embodiments, the plurality of anchoring elements 25 may be configured to prevent dislodgement and/or ejection of the occlusive implant 20 from the target site.

In some embodiments, the occlusive implant 20 and/or the expandable framework 22 may include a proximal hub 24 and a distal hub 26. The longitudinal axis 21 of the expandable framework 22 may extend from the proximal hub 24 to the distal hub 26. In at least some embodiments, the proximal hub 24 and/or the distal hub 26 may be centered on and/or coaxial with the longitudinal axis 102. The plurality of interconnected struts may be joined together at and/or fixedly attached to the proximal hub 24 and/or the distal hub 26. In some embodiments, the proximal hub 24 and/or the distal hub 26 may be fixedly attached to the expandable framework 22 and/or the plurality of interconnected struts, such as by welding, adhesive bonding, brazing, soldering, etc. The proximal hub 24 may be configured to releasably connect, couple, and/or attach the occlusive implant 20 and/or the expandable framework 22 to the distal end 32 of the core wire 30 (e.g., FIGS. 1-2). In some embodiments, the proximal hub 24 may include internal threads configured to rotatably and/or threadably engage external threads formed on and/or at the distal end 32 of the core wire 30. Other configurations for releasably securing the occlusive implant 20 to the core wire 30 are also contemplated.

In some embodiments, the occlusive implant 20 may optionally include an occlusive covering 28 connected to, disposed on, disposed over, disposed about, and/or disposed radially outward of a proximal portion of the expandable framework 22 and/or the plurality of interconnected struts. In some embodiments, the occlusive covering 28 may be attached to the proximal hub 24 and/or may be attached to the expandable framework at the proximal hub 24. In some embodiments, the occlusive covering 28 may extend radially outward from and/or may extend distally from the proximal hub 24. In some embodiments, the occlusive covering 28 may be attached and/or secured to the expandable framework 22 at a plurality of discrete locations. In some embodiments, one or more of the plurality of anchoring elements 25 may extend through the occlusive covering 28. In some embodiments, the one or more of the plurality of anchoring elements 25 extending through the occlusive covering 28 may attach and/or secure the occlusive covering 28 to the expandable framework 22.

In some embodiments, the occlusive covering 28 may include a membrane, a fabric, a mesh, a tissue clement, or another suitable construction. In some embodiments, the occlusive covering 28 may be porous. In some embodiments, the occlusive covering 28 may be non-porous. In some embodiments, the occlusive covering 28 may be permeable or impermeable to blood and/or other fluids, such as water. In some embodiments, the occlusive covering 28 may be designed, sized, and/or configured to prevent thrombus and/or embolic material from passing out of the left atrial appendage into the left atrium and/or the patient's bloodstream. In some embodiments, the occlusive covering 28 (e.g., the membrane, the fabric, or the tissue element, etc.) promotes endothelization after implantation, thereby effectively and/or permanently removing the target site (e.g., the left atrial appendage, etc.) from the patient's circulatory system. Some suitable, but non-limiting, examples of materials for the occlusive covering 28 are discussed below.

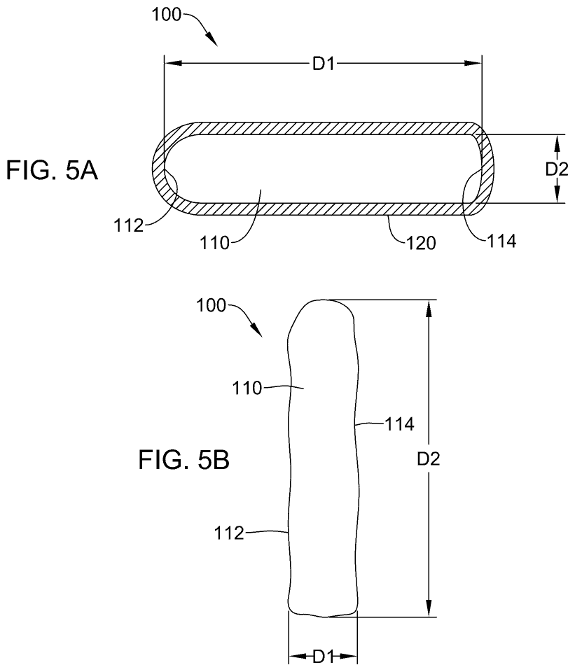

FIGS. 5A and 5B are cross-sectional views of an occlusive implant 100 according to the disclosure for use in occluding a body cavity, such as an aneurysm or LAA. The occlusive implant 100 may contain an expandable foam member 110 in a compressed configuration inside a layer of bioresorbable or biodegradable material 120 that temporarily constrains or holds the expandable foam member in the compressed configuration for a first time period, after which the biodegradable capsule degrades, allowing the expandable foam member to expand into an expanded configuration. In some embodiments, the expandable foam member 110 may be made of a shape memory polymer which may change from the compressed shape to the preset expanded shape in response to stimulus in the form of moisture, heat, change in pH, or electricity. The biodegradable material may be in the form of a biodegradable capsule 120 that protects the expandable foam member 110 from the stimuli until it is ready to expand in the anatomy. The biodegradable capsule 120 may cover the entirety of the outer surface of the expandable foam member 110. The biodegradable capsule 120 allows the expandable foam member 110 to be loaded into a delivery device, sterilized, and delivered to the desired anatomical location before fully expanding. The biodegradable capsule 120 may begin degrading during the implantation process, such as upon exposure to water or other fluids while being loaded into the delivery catheter just prior to delivery, during delivery through the body, and upon exposure to blood when implanted in the body. The biodegradable capsule 120 completes its degradation after being implanted in the body. The first time period, during which the biodegradable material 120 temporarily constrains or holds the expandable foam member in the compressed configuration, may be a predetermined time period and the thickness and composition of the biodegradable capsule 120 may be designed to achieve a desired level of degradation after the predetermined first time period. The desired level of degradation may be partial or complete. The first time period may be 30 seconds to 5 hours. In some embodiments, the time period may be less than 5 minutes, for example 30 seconds to 4 minutes. In some embodiments, the thickness of the biodegradable capsule 120 may be between 0.02 mm and 2 mm, for example 0.025 mm to 1 mm. The biodegradable capsule 120 of these thicknesses may be made of polyethylene glycol (PEG), gelatin, cellulose, starch, or combinations thereof.

In some embodiments, when in the compressed configuration, the expandable foam member 110 is elongate with a first end 112, a second opposing end 114, a first dimension D1 extending between the first and second opposing ends 112, 114, and a second dimension D2 transverse to the first dimension D1, as shown in FIG. 5A. The first and second dimensions D1, D2 are taken of just the expandable foam member 110, not including the biodegradable capsule 120. The first dimension D1 may be longer than the second dimension D2. In some examples, when the biodegradable capsule 120 has completely degraded and the expandable foam member 110 is in the fully expanded configuration, the first dimension D1 may be shorter than the second dimension D2, as shown in FIG. 5B. When in the compressed configuration (FIG. 5A), the first dimension D1 may be between 5 mm and 40 mm and the second dimension D2 may be between 1 mm and 10 mm, and when in the expanded configuration (FIG. 5B), the first dimension D1 may be between 5 mm and 15 mm and the second dimension D2 may be between 15 mm and 35 mm, producing a collapsibility ratio of at least 10 times (e.g., at least 10:1). The collapsibility ratio may be achieved with a variety of different dimensions. For example, the 10:1 ratio may be in one direction only, with D2 expansion only and no change to D1 when the implant 100 moves from the compressed to expanded configuration. In other examples, one of D1 or D2 may contract while the other dimension expands when the implant moves from the compressed to expanded configuration. In other embodiments, such as when it is desired for the implant 100 to be inserted into and substantially fill the LAA when in the expanded configuration, D1 in the expanded configuration may be up to 75 mm and D2 may be up to 60 mm. These dimensions may also be achieved with a plurality of separate implants 100. In addition to the substantially stadium shaped implant 100 shown in FIGS. 5A and 5B, the implant 100 may be oval, circular, polygonal, or have a non-uniform shape, such as a star or having non-uniform protrusions or lobes.

Other configurations are also contemplated, including those in which the first and second dimensions D1, D2 may be substantially the same in either or both of the compressed and expanded configurations, resulting in the ratio of D1 to D2 remaining substantially the same in the compressed and expanded configurations. In some embodiments, instead of the single, monolithic implant 100, multiple separate occlusive implants 100 may be used to fill the desired space. It will be understood that the dimensions described in association with the above figure are illustrative only, and that other dimensions of devices are contemplated. This embodiment may be particularly suited for delivery through a delivery sheath and implantation in and occlusion of the LAA, as will be discussed below.

In another embodiment of occlusive implant 200, the biodegradable capsule 220 may include at least one aperture 226 extending through the wall 228 defining the biodegradable capsule 220. The aperture 226 extends completely through the wall 228 such that the expandable foam member 210 is exposed to the environment outside of the biodegradable capsule 220. The one or more aperture 226 may be placed strategically to leave certain regions of the expandable foam member 210 unprotected to encourage expansion behavior in a specific location or to initiate the shape change early but prevent complete expansion for a period of time.

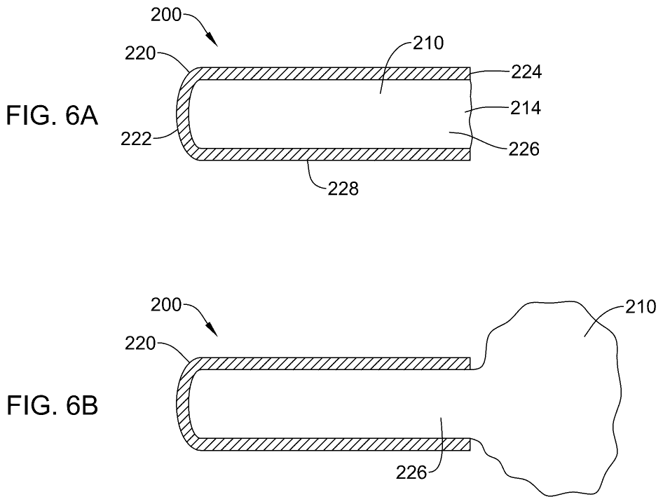

In the embodiment shown in FIGS. 6A and 6B, when in the compressed configuration, the expandable foam member 210 and the biodegradable capsule 220 are elongate, and the biodegradable capsule 220 has a first end 222 and a second opposing end 224, and a single aperture 226 at the second end 224. The biodegradable capsule 220 thus covers all but the second end 214 of the expandable foam member 210, leaving the second end 214 of the expandable foam member devoid of the biodegradable capsule 220. When the occlusive implant 200 is delivered into the body, the expandable foam member 210 starts to expand out of the aperture 226 of the biodegradable capsule 220, as shown in FIG. 6B. In this embodiment, the expandable foam member 210 expands at least partially before any degradation of the biodegradable capsule 220. When an occlusive implant 200 with a single opening 226 is used in an LAA occlusion device, the single opening 226 may be at the distal end so the expandable foam member 210 starts to expand distally and radially, which may aid in securing the occlusive implant 200 within the proximal region of the LAA occlusion device.

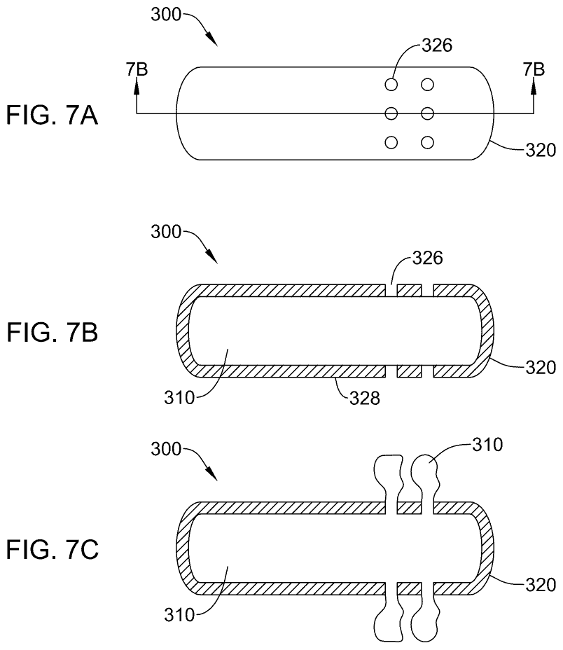

In other embodiments, the occlusive implant 300 may have a plurality of apertures extending through the biodegradable capsule 320. As shown in FIG. 7A, the biodegradable capsule 320 may have a plurality of spaced apart apertures 326. The apertures 326 may be disposed evenly along an entirety of the occlusive implant (not shown), or the apertures 326 may be disposed adjacent one end. In the embodiment shown in FIG. 7A, the apertures 326 are spaced apart circumferentially and are adjacent one end of the occlusive implant 300. As shown in the cross-sectional view in FIG. 7B, the plurality of apertures 326 extend completely through the wall 328 defining the biodegradable capsule 320. FIG. 7C illustrates the occlusive implant 300 immediately after being delivered into the body, with regions of the expandable foam member 310 expanding out through the plurality of apertures 326. The regions of expanding foam exiting the apertures 326 may form bumps or knobs that aid in securing the occlusive implant in place while the biodegradable capsule 320 degrades.

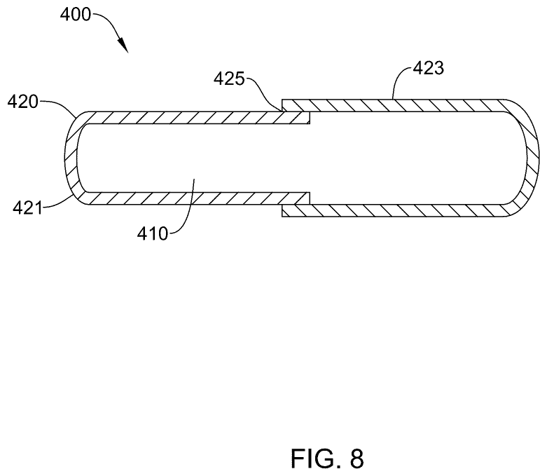

FIG. 8 illustrates an embodiment of occlusive implant 400 in which the expandable foam member 410 is enclosed by a biodegradable capsule 420 including a first section 421 and a second section 423 fixed to the first section 421 at a join 425. The join 425 may include a heat seal or weld or may include an adhesive. The first and second sections 421, 423 may be fitted over the expandable foam member 410 separately, and then the sections may be joined. The first section 421 and the second section 423 may be made of the same biodegradable material or the sections may be made of different biodegradable materials. In some embodiments the first and second sections 421, 423 may have different degradation rates. For example, the second section 423 may degrade partially or completely before the first section 421 begins to degrade.

In any of the above embodiments, the expandable foam member 110, 210, 310, 410 may be formed separately and then inserted into the biodegradable capsule 120, 220, 320, 420 which may then be sealed or crimped. In other embodiments, the biodegradable capsule 120, 220, 320, 420 may be sprayed onto the expandable foam member 110, 210, 310, 410, with any apertures masked off during the spraying procedure.

The biodegradable capsule 120, 220, 320, 430 may be made of one or more materials known to be biodegradable or bioresorbable. For example, the biodegradable capsule 120, 220, 320, 430 may be made of one or more of poly (ethylene glycol) (PEG), polyvinyl alcohol (PVA), polyurethane, polylactic acid (PLA), poly(lactide-co-glycolide) (PLGA), poly(ε-caprolactone) (PCL), sugar derivatives such as mannitol, salt, high surface area electrospun materials, and hydrogels. The biodegradable capsule 120, 220, 320, 430 may also be made of a copolymer of a hydrophobic component and a hydrophilic component. For example, a copolymer of poly (ethylene glycol) and polylactic acid may be used.

The occlusive implant 100 may be formed by compressing the expandable foam member 110 and subjecting it to e-beam sterilization, followed by enclosing the expandable foam member 110 with the biodegradable capsule 120 in a sterile environment. The selection of polymer coating and thickness for the biodegradable capsule 120 may be selected to minimize the penetration of ethylene oxide into the capsule 120. The expandable foam member 110 covered with the biodegradable capsule 120 may then be placed into a delivery system or integrated with an expandable framework or other devices. The fully assembled product can be sterilized using ethylene oxide as normal with reduced changes to the underlying shape memory polymer of the expandable foam member 110.

Additionally, selection of polymer coating and thickness for the biodegradable capsule 120 may be selected to control the degradation of the polymer such that expansion of the expandable foam member 110 would not begin for a controlled period of time. Once the expandable foam member 110 with biodegradable capsule 120 disposed thereover has been placed into a delivery system or integrated with an expandable framework or other devices, saline or fluoro dye may be flushed or injected around the device within a defined time limit without directly affecting the shape memory polymer expansion.

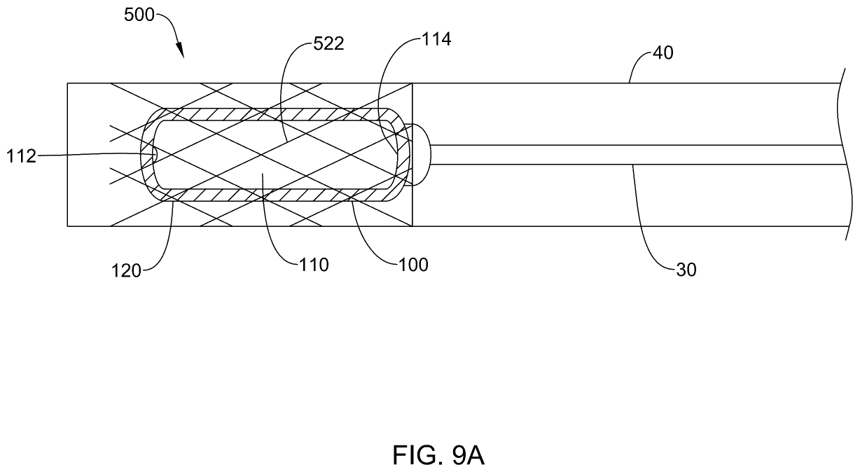

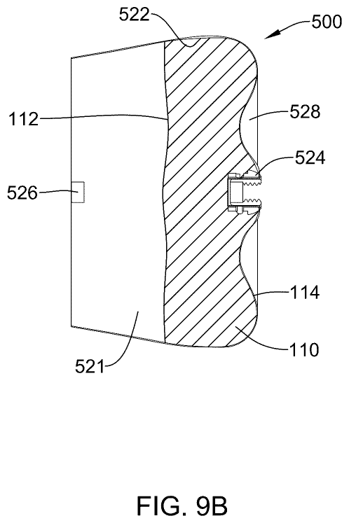

FIGS. 9A and 9B illustrate another embodiment in which an implant 500 further includes an expandable framework 522 configured to shift between a collapsed configuration (FIG. 9A) and an expanded configuration (FIG. 9B), where the expandable framework 522 defines an interior 521 into which the compressed occlusive implant 100 including the expandable foam member 110 covered with the biodegradable capsule 120 is disposed. The expandable framework 522 may be collapsed over the compressed occlusive implant 100 for delivery. The expandable framework 522 have a proximal hub 524 and a distal hub 526, may be self-expandable, made from a shape memory material, and may be delivered through a delivery sheath 40 using a core wire 30 as described above with regard to the occlusive implant system 10 shown in FIGS. 1-2. The proximal hub 524 may be removably coupled to a distal end of the core wire 30. When the expandable framework 522 has been deployed in the body and is in the expanded configuration, the biodegradable capsule 120 begins to degrade. When the biodegradable capsule 120 has completely degraded, the expandable foam member 110 fully expands to fill an entirety of at least a proximal region of the expandable framework 522, as shown in FIG. 9B. In some embodiments, the implant 500 further includes an occlusive covering 528 disposed on a proximal end region of the expandable framework 522. FIGS. 9A and 9B illustrate the change in shape of the expandable foam member 110 from the compressed configuration in FIG. 9A to the fully expanded configuration in FIG. 9B. The expandable foam member 110 may have the first end 112, second end 114 and similar dimensions D1 and D2 as described above with regard to the embodiment shown in FIGS. 5A and 5B.

In some embodiments, in the expanded configuration, the expandable foam member 110 may be configured to fill at least 40% of the interior 521 of the expandable framework 522 in the deployed configuration. In other embodiments, in the expanded configuration, the expandable foam member 110 may be configured to fill at least 80% of the interior 521 of the expandable framework 522 in the deployed configuration. In some embodiments, in the expanded configuration, the expandable foam member 110 may be configured to fill at least 85% of the interior 521 of the expandable framework 522 in the deployed configuration. In some embodiments, in the expanded configuration, the expandable foam member 110 may be configured to fill at least 90% of the interior 521 of the expandable framework 522 in the deployed configuration. In some embodiments, in the expanded configuration, the expandable foam member 110 may be configured to fill at least 95% of the interior 521 of the expandable framework 522 in the deployed configuration.

In some embodiments, the expandable foam member 110 may be configured to remain within the interior 521 of the expandable framework 522 permanently (e.g., the expandable foam member 110 is never removed from the interior 521 of the expandable framework 522 by the practitioner). In some embodiments, the expandable foam member 110 may be configured to be biodegradable over time. In some embodiments, the expandable foam member 110 may be configured to be biodegradable over at least 30 days' time. In some embodiments, the expandable foam member 110 may be configured to be biodegradable over at least 60 days' time. In some embodiments, the expandable foam member 110 may be configured to be biodegradable over at least 90 days' time. In some embodiments, the expandable foam member 110 may be configured to be biodegradable over at least 180 days' time. In some embodiments, the expandable foam member 110 may be configured to be biodegradable over at least 365 days' time. Other configurations are also contemplated.

In some embodiments, the expandable foam member 110 may be configured to prevent thrombus formation (e.g., within the left atrial appendage). In some embodiments, the expandable foam member 110 may include anti-thrombus medicament(s). In some embodiments, the expandable foam member 110 may be configured to absorb blood and/or bodily fluid(s). In some embodiments, the expandable foam member 110 may be configured to trap thrombus. In some embodiments, the expandable foam member 110 may be configured to promote tissue ingrowth and/or endothelization. Other configurations are also contemplated.

In at least some embodiments, the expandable foam member 110 may comprise and/or may be formed from a shape memory polymer and/or a shape memory foam. In at least some embodiments, the expandable foam member 110 may be configured as open celled foam. The shape memory polymer and/or the shape memory foam may have multiple geometric and/or mechanical properties when exposed to temperature, moisture, and/or chemical environments, and/or changes therein. In some embodiments, the shape memory polymer and/or the shape memory foam may have a collapsibility ratio that is high. The collapsibility ratio is a ratio between an expanded size and a collapsed size. In some examples, the collapsibility ratio of the shape memory polymer and/or the shape memory foam may be at least 5 times, at least 7 times, at least 8 times, at least 9 times, at least 10 times, at least 12 times, or more.

A method of occluding a body cavity using the occlusive implant 100 and expandable framework 522 may include the steps of inserting the expandable foam member 110 in a compressed configuration into a self-expandable framework 522, where the expandable foam member 110 is made of a shape memory polymer and covered by a biodegradable capsule 120. The biodegradable capsule 120 holds the expandable foam member 110 in the compressed configuration, and the self-expandable framework 522 is configured to shift between a collapsed configuration and an expanded configuration. The method next includes the step of compressing the self-expandable framework 522 to the collapsed configuration with the expandable foam member 110 inside. After compressing the self-expandable framework 522 around the expandable foam member 100, the method includes the steps of delivering the self-expandable framework 522 to a body cavity, expanding the self-expandable framework 522, and degrading the biodegradable capsule 120 in the presence of moisture and/or heat within the body cavity, thereby expanding the expandable foam member 110, where the expandable foam member 110 expands to fill at least a proximal region of the expanded self-expandable framework 522. In some embodiments, the body cavity is the LAA.

The materials that can be used for the various components of the system (and/or other elements disclosed herein) and the various components thereof disclosed herein may include those commonly associated with medical devices and/or systems. For simplicity purposes, the following discussion refers to the system. However, this is not intended to limit the devices and methods described herein, as the discussion may be applied to other elements, members, components, or devices disclosed herein, such as, but not limited to, the occlusive implant, the delivery sheath, the core wire, the expandable framework, the occlusive element, the capsule, the elongate fingers, the elongate strand, etc. and/or elements or components thereof.

In some embodiments, the system and/or components thereof may be made from a metal, metal alloy, polymer, a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304 and/or 316 stainless steel and/or variations thereof; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276R, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively bright image on a fluoroscopy screen or another imaging technique (e.g., ultrasound, etc.) during a medical procedure. This relatively bright image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, the system and/or components thereof may include a fabric material. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or components thereof may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni—Co—Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or components thereof may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. An occlusive implant comprising:
an expandable foam member in a compressed configuration, the expandable foam member made of a shape memory polymer; and
a biodegradable capsule disposed over the expandable foam member, the biodegradable capsule holding the expandable foam member in the compressed configuration for a first time period, after which the biodegradable capsule degrades, allowing the expandable foam member to expand into an expanded configuration.

2. The occlusive implant of claim 1, wherein the biodegradable capsule covers an entirety of an outer surface of the expandable foam member.

3. The occlusive implant of claim 1, wherein the biodegradable capsule includes at least one aperture extending through a wall of the biodegradable capsule.

4. The occlusive implant of claim 3, wherein when in the compressed configuration, the expandable foam member and the biodegradable capsule are elongate with first and second opposing ends, wherein the biodegradable capsule covers all but the second end of the expandable foam member.

5. The occlusive implant of claim 3, wherein when in the compressed configuration, the expandable foam member and the biodegradable capsule are elongate with first and second opposing ends, wherein the biodegradable capsule includes a plurality of spaced apart apertures disposed adjacent the first end.

6. The occlusive implant of claim 1, wherein the first time period is a preset time period.

7. The occlusive implant of claim 6, wherein the preset time period is 30 seconds to 4 minutes.

8. The occlusive implant of claim 1, wherein when in the compressed configuration, the expandable foam member is elongate with first and second opposing ends, the expandable foam member having a first dimension extending between the first and second opposing ends, and a second dimension transverse to the first dimension, the first dimension being longer than the second dimension, wherein when the expandable foam member is in the expanded configuration, the first dimension is shorter than the second dimension.

9. The occlusive implant of claim 8, wherein when in the expanded configuration the first dimension is between 3-15 mm and the second dimension is between 20-35 mm.

10. The occlusive implant of claim 1, wherein the biodegradable capsule is made of one or more materials selected from the group consisting of poly(ethylene glycol), polyvinyl alcohol, polyurethane, polylactic acid, poly(lactide-co-glycolide), poly(ε-caprolactone), sugar derivatives, salt, high surface area electrospun materials, and hydrogels.

11. The occlusive implant of claim 1, wherein the biodegradable capsule is made of a copolymer of a hydrophobic component and a hydrophilic component.

12. The occlusive implant of claim 11, wherein the copolymer includes poly(ethylene glycol) and polylactic acid.

13. The occlusive implant of claim 1, wherein the biodegradable capsule incudes a first section and a second section sealed to the first section.

14. The occlusive implant of claim 1, further comprising an expandable framework configured to shift between a collapsed configuration and an expanded configuration, the expandable framework defining an interior, wherein the expandable foam member is disposed within the interior.

15. The occlusive implant of claim 14, wherein when the expandable framework is in the expanded configuration and the biodegradable capsule has degraded, the expandable foam member expands to fill an entirety of at least a proximal region of the expandable framework.

16. The occlusive implant of claim 14, further comprising an occlusive covering disposed on a proximal end region of the expandable framework.

17. An occlusive implant comprising:
an expandable foam member in a compressed configuration, the expandable foam member made of a shape memory polymer that expands when exposed to water, increased temperature, change in pH, or electrical stimulation; and
a biodegradable capsule disposed over the expandable foam member, the biodegradable capsule holding the expandable foam member in the compressed configuration for a first time period, after which the biodegradable capsule degrades, allowing the expandable foam member to expand into an expanded configuration;
wherein when in the compressed configuration, the expandable foam member is elongate with first and second opposing ends, the expandable foam member having a first dimension extending between the first and second opposing ends, and a second dimension transverse to the first dimension, the first dimension being longer than the second dimension, wherein when the expandable foam member is in the expanded configuration, the first dimension is shorter than the second dimension.

18. The occlusive implant of claim 17, further comprising an expandable framework configured to shift between a collapsed configuration and an expanded configuration, the expandable framework defining an interior, wherein the expandable foam member is disposed within the interior.

19. The occlusive implant of claim 18, wherein when the expandable framework is in the expanded configuration and the biodegradable capsule has degraded, the expandable foam member expands to fill an entirety of at least a proximal region of the expandable framework.

20. A method of occluding a body cavity, comprising:
inserting an expandable foam member in a compressed configuration into a self-expandable framework, the expandable foam member made of a shape memory polymer and covered by a biodegradable capsule, the biodegradable capsule holding the expandable foam member in the compressed configuration, the self-expandable framework configured to shift between a collapsed configuration and an expanded configuration;
compressing the self-expandable framework to the collapsed configuration with the expandable foam member inside;
delivering the self-expandable framework to a body cavity;
expanding the self-expandable framework; and
degrading the biodegradable capsule in the presence of moisture and/or heat within the body cavity, thereby expanding the expandable foam member, wherein the expandable foam member expands to fill at least a proximal region of the expanded self-expandable framework.
